(19) United States
(12) Patent Application Publication
(10) Pub. No.: US 2015/0297181 A1
(43) Pub. Date: Oct. 22, 2015

(54) ULTRASOUND PROBE

(71) Applicants: SAMSUNG MEDISON CO., LTD., Hongcheon-gun (KR); SAMSUNG ELECTRONICS CO., LTD., Suwon-si (KR)

(72) Inventors: Saidmurod AKRAMOV, Hongcheon-gun (KR); Won-hee LEE, Hongcheon-gun (KR); Young-mun CHO, Hongcheon-gun (KR)

(21) Appl. No.: 14/665,990

(22) Filed: Mar. 23, 2015

(30) Foreign Application Priority Data
Apr. 22, 2014 (KR) 10-2014-0048203

(51) Int. Cl. A61B 8/00

(52) U.S. Cl. CPC A61B 8/4444; A61B 8/4483

(57) ABSTRACT

An ultrasound probe includes a transducer which generates ultrasound waves; an acoustic lens which focuses the ultrasound waves; a lens coating layer formed on at least a portion of an outer surface of the acoustic lens by mixing polymer particles with nanoparticles; and a housing which accommodates the transducer.

### RELATED APPLICATIONS

This application claims the benefit of Korean Patent Application No. 10-2014-0048203, filed on Apr. 22, 2014, in the Korean Intellectual Property Office, the disclosure of which is incorporated herein in its entirety by reference.

### BACKGROUND

1. Field

One or more embodiments of the present invention relate to ultrasound probes.

More particularly, one or more embodiments of the present invention relate to an ultrasound probe having a decreased surface frictional force and an increased durability.

2. Description of the Related Art

Ultrasound diagnosis apparatuses transmit an ultrasound signal generated by a transducer of a probe to an object and receive information regarding an ultrasound echo signal reflected from the object, thereby obtaining an image of a part inside the object. In particular, ultrasound diagnosis apparatuses are used for medical purposes, such as observation of the inside of an object, detection of foreign substances inside the object, and diagnosis of damage thereto. Such ultrasound diagnosis apparatuses have various advantages, including stability, real-time display, and safety because there is no exposure to radiation compared to X-ray apparatuses, and thus, the ultrasound diagnosis apparatuses are commonly used together with other image diagnosis apparatuses.

A transducer included in a probe includes an acoustic lens that generates and focuses ultrasound waves, which are acoustic energy. In general, an acoustic lens is formed of a material having high frictional resistance.

For ultrasound diagnosis, the probe needs to contact a portion of the skin of a patient, that is, an object, in order to scan the object. As described above, due to the high frictional resistance of the acoustic lens, lens abrasion quickly progresses, leading to a decrease in the durability of the probe. Furthermore, due to the high frictional resistance of the acoustic lens, when the probe is moved while in contact with the skin of a patient, a user should grip the probe tightly and the patient may feel uncomfortable.

Accordingly, there is a demand for an apparatus and method for easily scanning a patient when a probe scans the patient while in contact with the skin of the patient.

### SUMMARY

One or more embodiments of the present invention include an ultrasound probe capable of easily performing a scan.

One or more embodiments of the present invention also include an ultrasound probe having an increased durability.

Additional aspects will be set forth in part in the description which follows and, in part, will be apparent from the description, or may be learned by practice of the presented embodiments.

According to one or more embodiments of the present invention, an ultrasound probe includes a transducer which generates ultrasound waves; an acoustic lens which focuses the ultrasound waves; a lens coating layer formed on at least a portion of an outer surface of the acoustic lens by mixing polymer particles with nanoparticles; and a housing which accommodates the transducer.

The lens coating layer may have a lower friction coefficient than the acoustic lens.

The nanoparticles may be formed of a metal oxide.

The lens coating layer may be formed by mixing the polymer particles with silver (Ag) nanoparticles.

The lens coating layer may be formed by mixing the polymer particles with at least one selected from copper nanoparticles, titanium nanoparticles, and magnesium nanoparticles.

The nanoparticles may constitute 1% to 20% of the lens coating layer.

The nanoparticles may each have a diameter of 1 nm to 500 nm.

The lens coating layer may be a stack of a plurality of composite polymer layers.

The lens coating layer may be formed on at least a portion of the outer surface of the acoustic lens via deposition within a chamber in a vacuum state.

The transducer may include a piezoelectric element unit which generates the ultrasound waves in response to an electrical signal; a matching layer which changes an acoustic impedance of the ultrasound waves generated by the piezoelectric element unit; and a sound absorbing layer which absorbs ultrasound waves that are not transmitted toward an object from among the ultrasound waves generated by the piezoelectric element unit.

### BRIEF DESCRIPTION OF THE DRAWINGS

These and/or other aspects will become apparent and more readily appreciated from the following description of the embodiments, taken in conjunction with the accompanying drawings in which:

FIGS. 1A and 1B are views of an ultrasound probe according to an embodiment of the present invention;

FIGS. 2A and 2B are longitudinal sectional views of an ultrasound probe according to another embodiment of the present invention;

FIG. 3 is a view of the ultrasound probe illustrated in FIG. 2;

FIG. 4 is a block diagram for explaining the manufacture of the ultrasound probe illustrated in FIG. 1, according to an embodiment of the present invention;

FIG. 5 is a table for explaining physical properties of the ultrasound probe illustrated in FIG. 1, according to an embodiment of the present invention;

FIG. 6 is a table for explaining a reduction in the friction coefficient of the ultrasound probe illustrated in FIG. 1, according to an embodiment of the present invention;

FIG. 7 is a bar graph for explaining the durability of the ultrasound probe illustrated in FIG. 1, according to an embodiment of the present invention;

FIG. 8 is a view for explaining the acoustic properties of the ultrasound probe illustrated in FIG. 1, according to an embodiment of the present invention; and

FIG. 9 is a block diagram of an ultrasound diagnosis apparatus including an ultrasound probe, according to an embodiment of the present invention.

### DETAILED DESCRIPTION

Embodiments of the present invention are described in detail herein with reference to the accompanying drawings so that this disclosure may be easily performed by one of ordinary skill in the art to which the present invention pertain. The invention may, however, be embodied in many different forms and should not be construed as being limited to the embodiments set forth herein. In the drawings, parts irrelevant to the description are omitted for simplicity of explanation, and like numbers refer to like elements throughout.

Throughout the specification, when an element is referred to as being “connected” or “coupled” to another element, it can be directly connected or coupled to the other element, or can be electrically connected or coupled to the other element with intervening elements interposed therebetween. In addition, the terms “comprises” and/or “comprising” or “includes” and/or “including” when used in this specification, specify the presence of stated elements, but do not preclude the presence or addition of one or more other elements. In addition, terms such as “ . . . unit”, “ . . . module”, or the like refer to units that perform at least one function or operation, and the units may be implemented as hardware or software or as a combination of hardware and software.

Throughout the specification, an “ultrasound image” refers to an image of an object that is acquired using ultrasound waves. Throughout the specification, “object” may include a person, animal, or a part of a person or animal. For example, the object may include at least one selected from an organ (for example, the liver, the heart, the womb, the brain, a breast, or the abdomen) and a blood vessel. The object may be a phantom. The phantom means a material having a density, an effective atomic number, and a size that are approximately the same as those of a living thing.

Throughout the specification, “user” refers to a medical professional, such as a doctor, a nurse, a medical laboratory technologist, and an engineer who repairs a medical apparatus, but the user is not limited thereto.

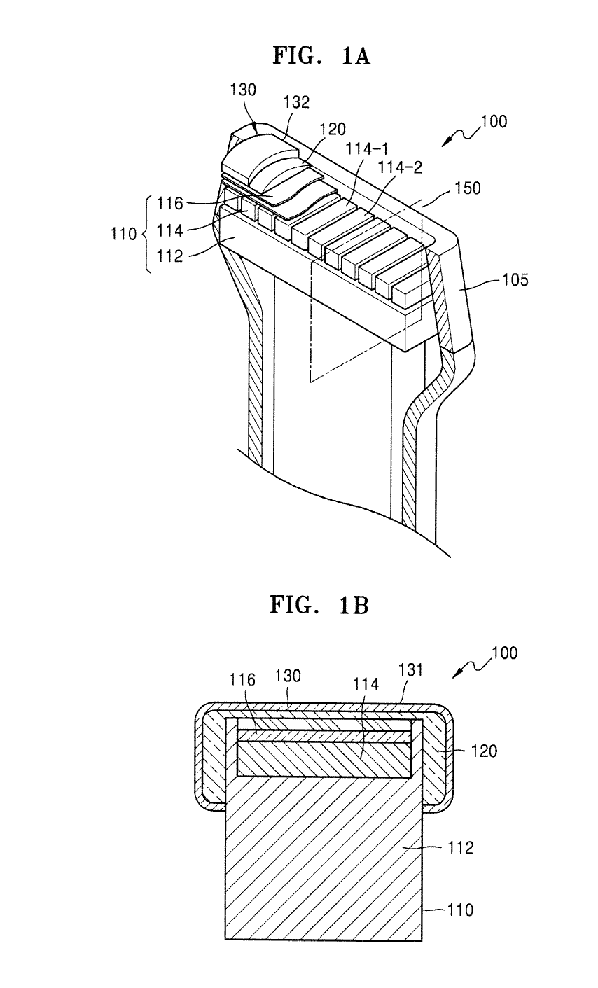

FIGS. 1A and 1B are views of an ultrasound probe 100 according to an embodiment of the present invention. In detail, FIG. 1A is a cutaway view of the ultrasound probe 100. FIG. 1B is a longitudinal sectional view of the ultrasound probe 100.

The ultrasound probe 100 includes a plurality of transducers 110. Each of the transducers 110 vibrates according to a received electrical signal, generates ultrasound waves, which are acoustic energy, and transmits the ultrasound waves to an object. Each of the transducers 110 receives an ultrasound echo signal, which is an ultrasound signal reflected from the object.

The ultrasound probe 100 may be used not only in ultrasound diagnosis apparatuses for diagnosing a disease of a patient but also in various ultrasound apparatuses related to probing.

Referring to FIG. 1A, the ultrasound probe 100 includes the transducers 110, an acoustic lens 120, a lens coating layer 130, and a housing 105.

The transducers 110 generate ultrasound waves. In detail, the transducers 110 generate ultrasound waves according to a received voltage.

The acoustic lens 120 focuses the ultrasound waves generated by the transducers 110. Accordingly, the acoustic lens 120 applies focused ultrasound waves to an object.

The lens coating layer 130 is formed on at least a portion of an outer surface of the acoustic lens 120 and is formed of a material mixed with nanoparticles.

The housing 105 forms the body of the ultrasound probe 100. In other words, as illustrated in FIG. 1B, the transducers 110 are accommodated within the housing 105.

In detail, in response to an alternating voltage, the transducers 110 may generate ultrasound waves due to vibration of a piezoelectric material included therein. In detail, the transducers 110 may include a sound absorbing layer 112, a piezoelectric element unit 114, and a matching layer 116.

In detail, the piezoelectric element unit 114 includes at least one piezoelectric element, namely, piezoelectric elements 114-1 and 114-2, which transform an electrical signal to an acoustic signal or vice versa. The piezoelectric elements 114-1 and 114-2 may be formed by splitting a piezoelectric material. The piezoelectric element unit 114 may receive an electrical signal via both ends thereof. For example, electrodes may respectively be formed on both ends of the piezoelectric element unit 114, and a voltage may be applied to both electrodes. The electrodes formed on both ends of the piezoelectric element unit 114 are not illustrated in FIG. 1A.

For example, as illustrated in FIG. 1A, the piezoelectric element unit 114 may be manufactured by dicing a piezoelectric material extending in a length direction. However, the manufacture of the piezoelectric elements 114-1 and 114-2 is not limited to this dicing method, and the piezoelectric elements 114-1 and 114-2 may be manufactured using various other methods, such as, a method of pressing a piezoelectric element by using a metal mold.

Examples of the piezoelectric material used to form the piezoelectric element unit 114 may include, but are not limited to, piezoelectric ceramics, a single crystal material, and a composite piezoelectric material which is a compound of a polymer material and any of the aforementioned materials. The piezoelectric ceramics, the single crystal material, and the composite piezoelectric material cause a piezoelectric effect. The piezoelectric ceramics mechanically deform due to a voltage generated when being pressurized, and thus vibrate. Accordingly, when a voltage is applied to piezoelectric ceramics, the piezoelectric ceramics vibrate and thus ultrasound waves may be generated.

The matching layer 116 is disposed on a front surface of the piezoelectric element unit 114. The matching layer 116 changes an acoustic impedance of the ultrasound waves generated by the piezoelectric element unit 114 in stages so that the acoustic impedance of the ultrasound waves is approximate to an acoustic impedance of the object. The front surface of the piezoelectric element unit 114 may be a surface that is closest to the object from among the surfaces of the piezoelectric element unit 114 when ultrasound waves are applied to the object, and a rear surface thereof may be a surface opposite to the front surface. The matching layer 116 is also called an acoustic matching layer.

The matching unit 116 may extend lengthwise along the front surface of the piezoelectric element unit 114, but one or more embodiments of the present invention are not limited thereto. The matching unit 116 may be partially formed on the piezoelectric element unit 114. In the present embodiment, the matching unit 116 has a single-layered structure. However, in another embodiment, the matching unit 116 may have a multi-layered structure.

The sound absorbing layer 112 may support the piezoelectric element unit 114 at the back surface of the piezoelectric element unit 114, and absorb ultrasound waves that are transmitted toward the back surface of the piezoelectric element unit 114 and is thus not directly used in tests or diagnosis. The sound absorbing layer 112 may be formed in a length direction of the piezoelectric element unit 114 to have the same length as that of the piezoelectric element unit 114. The length direction may be a direction along the long edge of the piezoelectric element unit 114 as illustrated in FIG. 1A.

The sound absorbing layer 112 may include a plurality of electrodes for applying voltages to the piezoelectric element unit 114. Since the electrodes are connected to the piezoelectric elements 114-1 and 114-2 of the piezoelectric element unit 114 in a one-to-one correspondence, the number of electrodes may be equal to that of piezoelectric elements 114-1 and 114-2.

The acoustic lens 120 is disposed on the front surface of the transducer 110 and focuses the ultrasound waves generated by the piezoelectric element unit 114. The acoustic lens 120 may be formed of a material such as silicon rubber having an acoustic impedance that is similar to that of the object. A central portion of the acoustic lens 120 may be convex or flat. The acoustic lens 120 may have various shapes according to designs of manufacturers.

The lens coating layer 130 is coated on a portion of the acoustic lens 120. In detail, the lens coating layer 130 may cover the entire front surface of the acoustic lens 120. The lens coating layer 130 may cover a portion of the acoustic lens 120 that contacts the skin of a patient. The lens coating layer 130 will now be described in detail with reference to FIG. 1B.

FIG. 1B illustrates a cross-section 150 of the ultrasound probe 100 illustrated in FIG. 1A, in greater detail. In FIG. 1B, only the transducer 110, the acoustic lens 120, and the lens coating layer 130 are illustrated. FIG. 1B illustrates a case where the lens coating layer 130 is formed to cover the front surface of the acoustic lens 120.

The lens coating layer 130 may be formed of a composite polymer layer having a lower friction coefficient than the acoustic lens 120. The composite polymer layer is formed by mixing nanoparticles and polymer particles.

In detail, each nanoparticle may have a diameter of about 1-500 nm. Each polymer particle may also have a diameter of 1-500 nm.

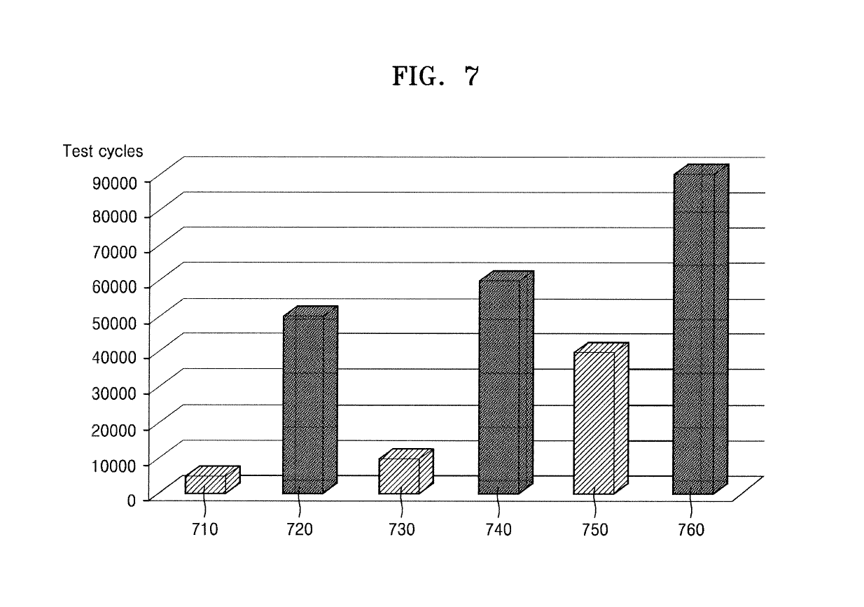

The composite polymer layer used to form the lens coating layer 130 may have a thickness of about 1-20 um. In detail, the composite polymer layer used to form the lens coating layer 130 may have a thickness of about 20 um. When the composite polymer layer is formed to have a thickness of about 20 um, it may not affect the acoustic characteristics of the ultrasound probe 100, and at the same time the durability and resistance to wear of the ultrasound probe 100 that will be described later with reference to FIG. 7 may be increased.

When using a conventional ultrasound probe during an ultrasound test, an acoustic lens may directly contact the skin of a patient. The acoustic lens is formed of a material having a high friction coefficient, for example, silicon rubber. Accordingly, when the acoustic lens scans the patient while in contact with the skin of the patent, a scan is not smoothly performed. In addition, due to the high friction coefficient of the acoustic lens, the surface of the acoustic lens quickly wears. This abrasion of the surface of the acoustic lens may lead to a decrease in the durability of the conventional ultrasound probe.

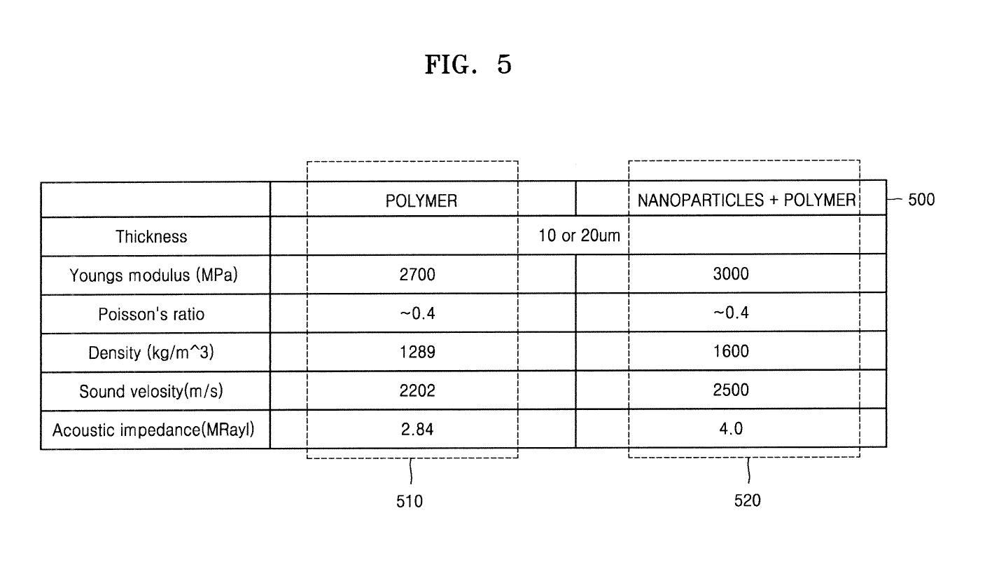

In the ultrasound probe 100 according to the present embodiment, an upper surface of the acoustic lens 120 that contacts the skin of a patient is coated with the lens coating layer 130, which is formed by mixing nanoparticles and polymer particles, thereby reducing surface abrasion of the acoustic lens 120 and increasing the durability of the ultrasound probe 100. The physical properties of the ultrasound probe 100 including abrasion resistance will be described in more detail later with reference to FIGS. 5-7.

In detail, the polymer used to form the lens coating layer 130 may be parylene. Alternatively, the polymer used to form the lens coating layer 130 may be a fluorine polymer, an acryl polymer, a urethane polymer, a silicon polymer, or the like.

Examples of the fluorine polymer may include Ploy Tetra fluoro Ethylene (PTFE), Fluorinated ethylene propylene copolymer (FEP), and Ethylene-tetrafluoroethylene (ETFE).

The aforementioned polymers may each have a friction coefficient of about 0.83. Thus, the lens coating layer 130 formed of a polymer may have a friction coefficient of about 0.83. The friction coefficient of this polymer may be about 10-30% lower than that of silicon rubber used to form the acoustic lens 120. Accordingly, when the acoustic lens 120 is coated with a polymer, surface friction resistance between the ultrasound probe 100 and the skin of a patient may be reduced.

Although the lens coating layer 130 is a single layer in FIG. 1B, the lens coating layer 130 may be a stack of a plurality of composite polymer layers. For example, after the upper surface of the acoustic lens 120 is coated with a first composite polymer layer formed of a mixture of PTFE, which is a fluorine polymer, and nanoparticles, an upper surface of the first composite polymer layer may be coated with a second composite polymer layer formed of a mixture of parylene and nanoparticles.

In detail, the polymer used to form the lens coating layer 130 may have a density of about 0.6 to 1.5 g/cm³.

The polymer used to form the lens coating layer 130 may have hardness of about R75 to R90 on a Rockwell scale.

The thickness of the composite polymer layer used to form the lens coating layer 130 may vary according to wavelengths (lambda) of piezoelectric elements included in the piezoelectric element unit 114 of the transducer 110. In detail, the thickness of the composite polymer layer may be about 1/50 to 1/25 the wavelength of a piezoelectric element.

The composite polymer layer used to form the lens coating layer 130 may have an acoustic impedance of about 2.7 MRayls or less. Accordingly, the acoustic impedance of the lens coating layer 130 may be matched to the acoustic impedance matching of a body of the patient. In other words, acoustic impedance matching to the body of a patient may be performed during an ultrasound scan, by using the composite polymer layer having an acoustic impedance of about 2.7 MRayls or less. In addition, by using the composite polymer layer having an acoustic impedance of about 2.7 MRayls or less, primary acoustic impedance matching may be performed by using the matching layer 116, and secondary acoustic impedance matching may be performed by using the composite polymer layer. Thus, the acoustic impedance of an ultrasound signal applied to the body of a patient may be made closer to that of the body of the patient, and thus a transmitting-receiving rate of the ultrasound signal may be increased.

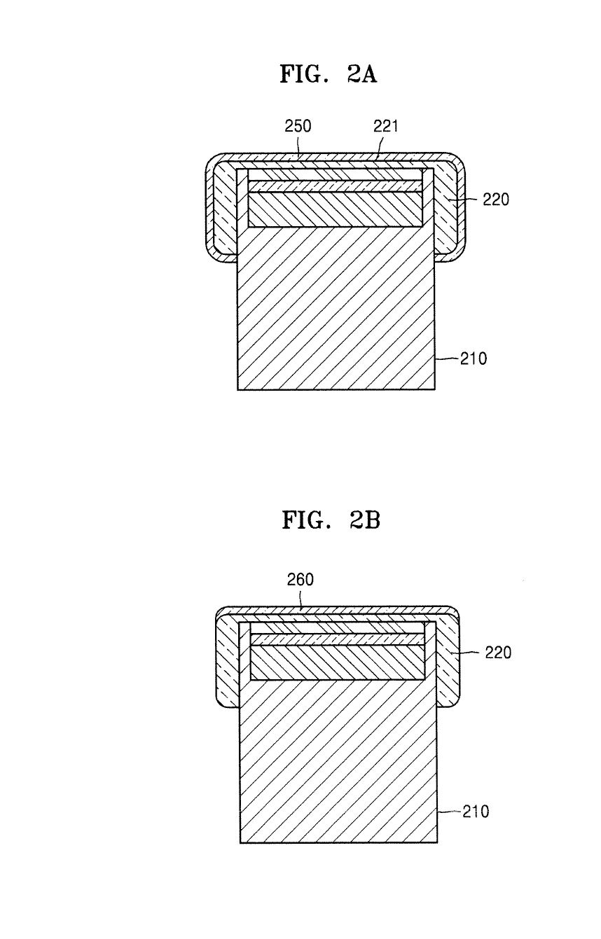

FIGS. 2A and 2B are longitudinal sectional views of an ultrasound probe according to another embodiment of the present invention.

A transducer 210, an acoustic lens 220, and a lens coating layer 260 of FIGS. 2A and 2B correspond to the transducer 110, the acoustic lens 120, and the lens coating layer 130 of FIGS. 1A and 1B, respectively. Thus, a repeated description thereof will be omitted.

Referring to FIG. 2A, an upper surface 221 of the acoustic lens 220 may be activated to form the lens coating layer 260. In detail, an adhesion promoter 250 may be coated on an upper surface 221 of the acoustic lens 220. The adhesion promoter 250 is used to promote adhesion between the acoustic lens 220 and a polymer layer, and may be coated on the upper surface 221 of the acoustic lens 220 before the acoustic lens 220 is coated with the polymer layer. For example, silane may be used as the adhesion promoter.

Referring to FIG. 2B, after the adhesion promoter 250 is removed, the upper surface 221 activated by the adhesion promoter 250 may be coated with the lens coating layer 260. Although the lens coating layer 260 is coated on a portion of the acoustic lens 220 in FIG. 2B, the lens coating layer 260 may be formed on the entire outer surface of the acoustic lens 220.

As described above, by activating the upper surface 221 of the acoustic lens 220 by using the adhesion promoter 250 and then forming the lens coating layer 260 on the activated upper surface 221, the lens coating layer 260 may be uniformly formed and may easily contact the acoustic lens 220.

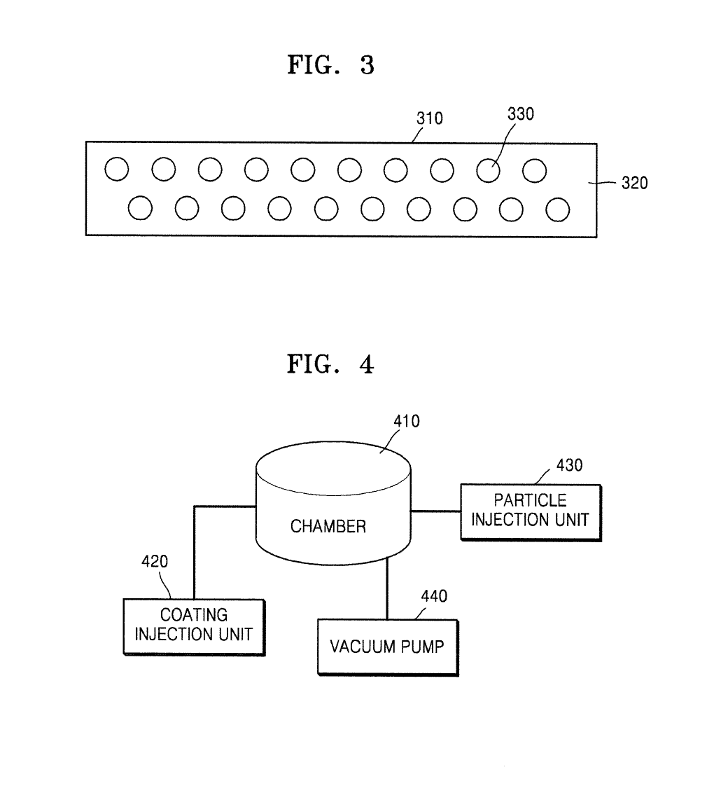

FIG. 3 is another view for the ultrasound probe 100. In detail, FIG. 3 is a view for explaining a composite polymer layer including nanoparticles. Since the lens coating layer 310 of FIG. 3 corresponds to the lens coating layer 130 of FIG. 1, a repeated description thereof will be omitted.

An upper surface of the lens coating layer 310 that contacts the skin of a patient is illustrated in FIG. 3. The upper surface of the lens coating layer 310 of FIG. 3 corresponds to a surface 132 of the lens coating layer 130 of FIG. 1 that contacts the skin of a patient. A case where the lens coating layer 310 is formed of a composite polymer layer in which nanoparticles 330 are mixed with a polymer will now be described.

The composite polymer layer 310 is formed by mixing nanoparticles 330 with a polymer 320 formed of polymer particles.

The nanoparticles 330 may be formed of a metal oxide. For example, the nanoparticles 330 may be silver (Ag) nanoparticles. Alternatively, the nanoparticles 330 may be nanoparticles formed of a metal such as copper (Cu), titanium (Ti), or magnesium (Mg). The aforementioned nanoparticles may each have a size of about 11-500 nm. While oxidizing, the aforementioned nanoparticles remove germs, such as bacteria, from a coating layer. Thus, the growth of germs or bacteria that may exist on a portion of an ultrasound probe that contacts the body of a patient may be suppressed.

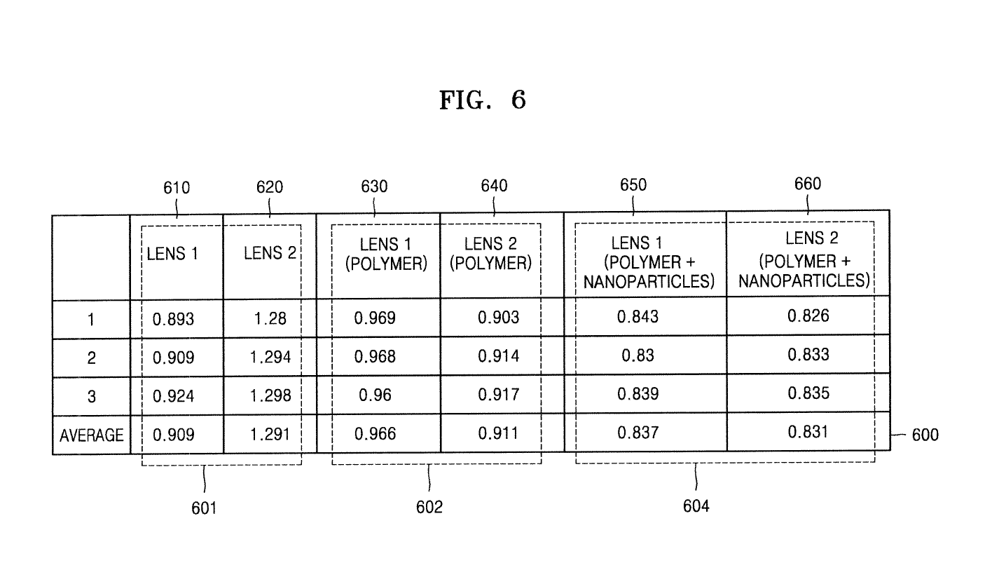

When the nanoparticles 330 are disposed on the polymer 320, durability or the like against friction, which is a mechanical property, may be improved. The durability improvement will be described in more detail later with reference to FIGS. 6 and 7.

The nanoparticles 330 may constitute about 1-20% of the composite polymer layer 310. Alternatively, when the nanoparticles 330 constitute 1-20% of the composite polymer layer 310 and each have a size of about 11-500 nm, they may not affect an acoustic property of the ultrasound probe 100, such as an acoustic impedance value, and at the same time the durability of the ultrasound probe 100 may be improved. In other words, even when the nanoparticles 330 are included in the composite polymer layer 310, an acoustic impedance of the composite polymer layer 310 may not be increased, and at the same time the durability of the ultrasound probe 100 may be improved and an antibacterial effect may be achieved.

FIG. 4 is a block diagram for explaining manufacturing of the ultrasound probe 100, according to an embodiment of the present invention.

Referring to FIG. 4, the lens coating layer 130 of the ultrasound probe 100 may be manufactured within a chamber 410.

The chamber 410 is a process chamber for coating the lens coating layer 130. A coating injection unit 420 injects the polymer particles that form the lens coating layer 130, into the chamber 410. A particle injection unit 430 injects the nanoparticles 330 into the chamber 410. A vacuum pump 440 keeps the chamber 410 in a vacuum state so that the polymer particles and the nanoparticles 300 may be coated on the acoustic lens 120 within vacuum conditions.

For example, the lens coating layer 130 may be formed on the upper surface of the acoustic lens 120 within the chamber 410 via chemical vapor deposition (CVD). Alternatively, the lens coating layer 130 may be formed using various other deposition methods.

FIG. 5 is a table for explaining physical properties of the ultrasound probe 100, according to an embodiment of the present invention.

FIG. 5 illustrates physical properties of the lens coating layer 130 of the ultrasound probe 100. In detail, FIG. 5 illustrates the physical properties of the composite polymer layer 310 described above with reference to FIG. 3. A case where the nanoparticles 330 of the composite polymer layer 310 are Ag nanoparticles and the polymer 320 thereof is formed of parylene particles will now be illustrated and described. In this case, the composite polymer layer 320 includes about 10% of Ag nanoparticles.

Referring to FIG. 5, a physical property table 500 includes physical properties 510 of a coating layer formed of only a polymer to cover the acoustic lens 120, and physical properties 520 of a composite polymer layer which is a coating layer formed of a polymer and nanoparticles. Hereinafter, a coating layer formed of only a polymer is referred to as a polymer layer, and a coating layer formed of polymer and nanoparticles is referred to as a composite polymer layer.

Referring to the physical property table 500, the thicknesses of the polymer layer and the composite polymer layer are all 10 um or 20 um.

With respect to a Young's modulus, which is a measure of the deformation degree of a material, the polymer layer has a value of 2700 MPa, and the composite polymer layer has a value of 3000 MPa.

With respect to a Poisson's ratio representing the deformation degree of a material, the polymer layer has a value of 0.4 or less, and the composite polymer layer also has a value of 0.4 or less.

The polymer layer has a density of 1289 kg/m³, and the composite polymer layer has a density of 1600 kg/m³.

A sound velocity in the polymer layer is 2202 m/s and a sound velocity in the composite polymer layer is 2500 m/s.

The polymer layer has an acoustic impedance of 2.84 MRayl and the composite polymer layer has an acoustic impedance of 4.0 MRayl.

FIG. 6 is a table for explaining a reduction in the friction coefficient of the ultrasound probe 100, according to an embodiment of the present invention.

Referring to FIG. 6, a friction coefficient table 600 includes a friction coefficient 601 of an acoustic lens, a friction coefficient 602 when an upper surface of the acoustic lens is coated with a polymer layer, and a friction coefficient 604 when the upper surface of the acoustic lens 120 is coated with the composite polymer layer 310. FIG. 6 shows results of three friction coefficient measurements for each case. FIG. 6 also includes measurements of the friction coefficients of two acoustic lenses formed of different kinds of silicon, namely, a lens 1 and a lens 2.

The friction coefficient of an acoustic lens was measured by rubbing the acoustic lens against foaming silicon having characteristics similar to those of the skin of a human body.

In detail, the friction coefficient table 600 includes a friction coefficient 610 of an uncoated lens 1, a friction coefficient 620 of an uncoated lens 2, a friction coefficient 630 of a lens 1 coated with a polymer layer, a friction coefficient 640 of a lens 2 coated with a polymer layer, a friction coefficient 650 of a lens 1 coated with the composite polymer layer 310, and a friction coefficient 660 of a lens 2 coated with the composite polymer layer 310.

Referring to the friction coefficient table 600, an average friction coefficient of the uncoated lens 1 is 0.909, an average friction coefficient of the lens 1 coated with the polymer layer is 0.966, and an average friction coefficient of the lens 1 coated with the composite polymer layer 310 is 0.837. Accordingly, the friction coefficient when the lens 1 is coated with the composite polymer layer 310 is the smallest, and the friction coefficient of the lens 1 coated with the composite polymer layer 310 was reduced by about 10% in comparison to that of the uncoated lens 1.

Referring to the friction coefficient table 600, an average friction coefficient of the uncoated lens 2 is 1.291, an average friction coefficient of the lens 2 coated with the polymer layer is 0.911, and an average friction coefficient of the lens 2 coated with the composite polymer layer 310 is 0.831. Accordingly, the friction coefficient when the lens 2 is coated with the composite polymer layer 310 is the smallest, and the friction coefficient of the lens 2 coated with the composite polymer layer 310 was reduced by about 30% in comparison to that of the uncoated lens 2.

As described above, in the ultrasound probe 100, since the acoustic lens 120 is coated with the composite polymer layer 130 or 310, the friction coefficient of the acoustic lens 120 may be effectively reduced compared with an existing acoustic lens, and thus an ultrasound scan may be easily performed and abrasion due to friction may be reduced.

FIG. 7 is a bar graph for explaining the durability of the ultrasound probe 100, according to an embodiment of the present invention.

FIG. 7 shows results of an abrasion experiment using a coated acoustic lens. In the abrasion experiment, whether the surface of the coated acoustic lens has been peeled off or damaged was determined repeatedly. In detail, in the abrasion experiment, the number of times a test was performed until a coating layer on an acoustic lens was peeled off or damaged was measured to thereby ascertain abrasivity of the coated acoustic lens. Performing the test one time denotes contacting the skin or a material similar to the skin once with the coated acoustic lens.

In detail, a bar 710 represents the number of tests performed until a polymer layer is formed of parylene as a polymer on the acoustic lens to have a thickness of 1 um is peeled off or damaged. A bar 720 represents the number of tests performed until the composite polymer layer 310 formed on the acoustic lens to have a thickness of 1 um is peeled off or damaged.

A bar 730 represents the number of tests performed until a polymer layer of parylene as a polymer coated on the acoustic lens to have a thickness of 5 um is peeled off or damaged. A bar 740 represents the number of tests performed until the composite polymer layer 310 formed on the acoustic lens to have a thickness of 5 um is peeled off or damaged.

A bar 750 represents the number of tests performed until a polymer layer formed of parylene as a polymer on the acoustic lens to have a thickness of 10 um is peeled off or damaged. A bar 760 represents the number of tests performed until the composite polymer layer 310 formed on the acoustic lens to have a thickness of 10 um is peeled off or damaged.

As shown in FIG. 7, in all cases where coating layers are formed to have thicknesses of 1 um, 5 um, and 10 um, the number of tests required until the coating layer is peeled off or damaged is overwhelmingly higher when the acoustic lens is coated with the composite polymer layer 310 than when the acoustic lens is coated with the polymer layer. In other words, when the acoustic lens is coated with the composite polymer layer 310, the durability of the acoustic lens may be greatly increased.

Although not shown in FIG. 7, when the composite polymer layer 310 is coated on the acoustic lens to have a thickness of 20 um, the number of tests performed is greater than when the composite polymer layer 310 is coated on the acoustic lens to have a thickness of 10 um. Accordingly, when the composite polymer layer 310 is coated on the acoustic lens to have a thickness of 20 um, the durability of the acoustic lens may be increased compared with when parylene coating is performed.

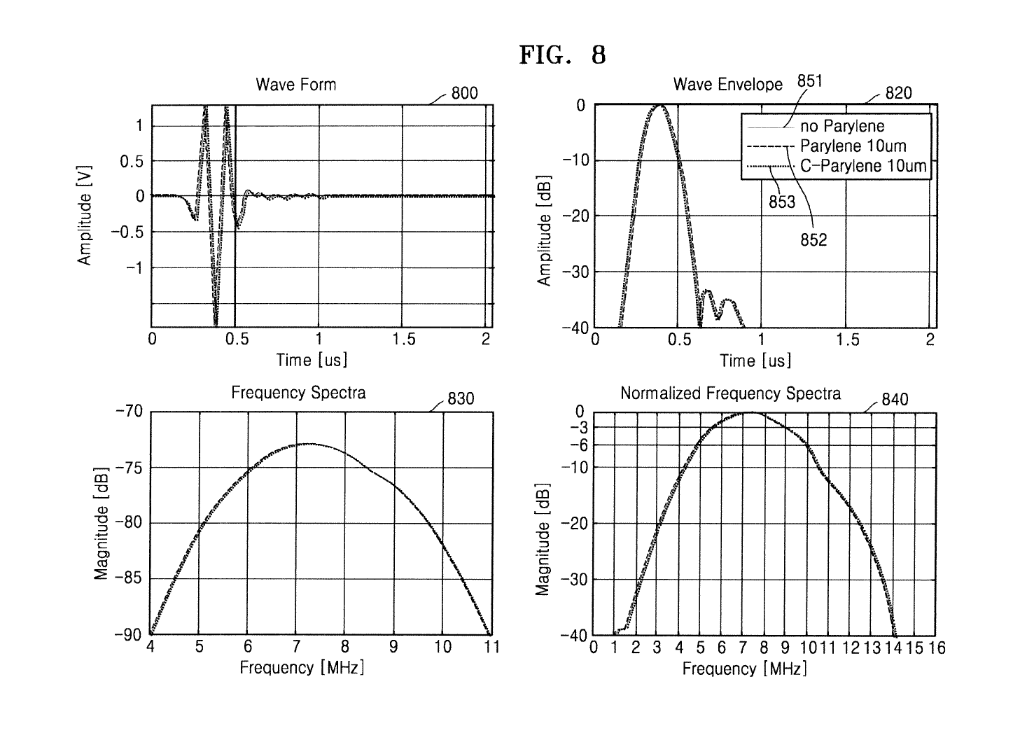

FIG. 8 is a view for explaining the acoustic properties of the ultrasound probe 100, according to an embodiment of the present invention.

Referring to FIG. 8, a graph 810 represents a wave form of ultrasound waves transmitted toward an object. In the graph 810, the x axis indicates time, and the y axis indicates amplitude of the ultrasound waves. A graph 820 represents a wave envelope of the ultrasound waves transmitted toward the object. In the graph 820, the x axis indicates time, and the y axis indicates the amplitude of the ultrasound waves. A graph 830 represents frequency spectra of the ultrasound waves transmitted toward the object. In the graph 830, the x axis indicates time, and the y axis indicates a magnitude of the ultrasound waves. A graph 840 represents normalized frequency spectra obtained by normalizing the ultrasound signal transmitted toward an object. In the graph 840, the x axis indicates time, and the y axis indicates the magnitude of the ultrasound waves. In the graphs 810, 820, 830, and 840, a solid line 851 represents a case where ultrasound waves are transmitted toward an object via an uncoated acoustic lens, and an equally-spaced dashed line 852 represents a case where ultrasound waves are transmitted toward the object via an acoustic lens coated with parylene as a polymer. An irregularly-spaced dotted line 853 represents a case where ultrasound waves are transmitted toward the object via an acoustic lens coated with the composite polymer layer 310. In FIG. 8, the equally-spaced dashed line 852 represents a case where parylene is coated to have a thickness of 10 um, and the irregularly spaced dotted line 853 represents a case where the composite polymer layer 310 is coated to have a thickness of 10 um.

As illustrated in the graphs 810, 820, 830, and 840 of FIG. 8, acoustic properties, such as a waveform, a wave envelope, frequency spectra, and normalized frequency spectra, are almost the same in all cases where ultrasound waves are transmitted toward the object via the uncoated acoustic lens, where ultrasound waves are transmitted toward the object via the acoustic lens coated with parylene, and where ultrasound waves are transmitted toward the object via the acoustic lens coated with the composite polymer layer 310. Although not illustrated in FIG. 8, even when the composite polymer layer 310 is coated to have a thickness of 20 um, acoustic properties, such as a wave form, a wave envelope, frequency spectra, and normalized frequency spectra, are not different from those when ultrasound waves are transmitted toward the object via the uncoated acoustic lens and when ultrasound waves are transmitted toward the object via the acoustic lens coated with parylene. Accordingly, when the composite polymer layer 310 is coated on the acoustic lens, it may not affect the acoustic properties, and at the same time the friction coefficient of the acoustic lens may be reduced and the durability thereof may be increased.

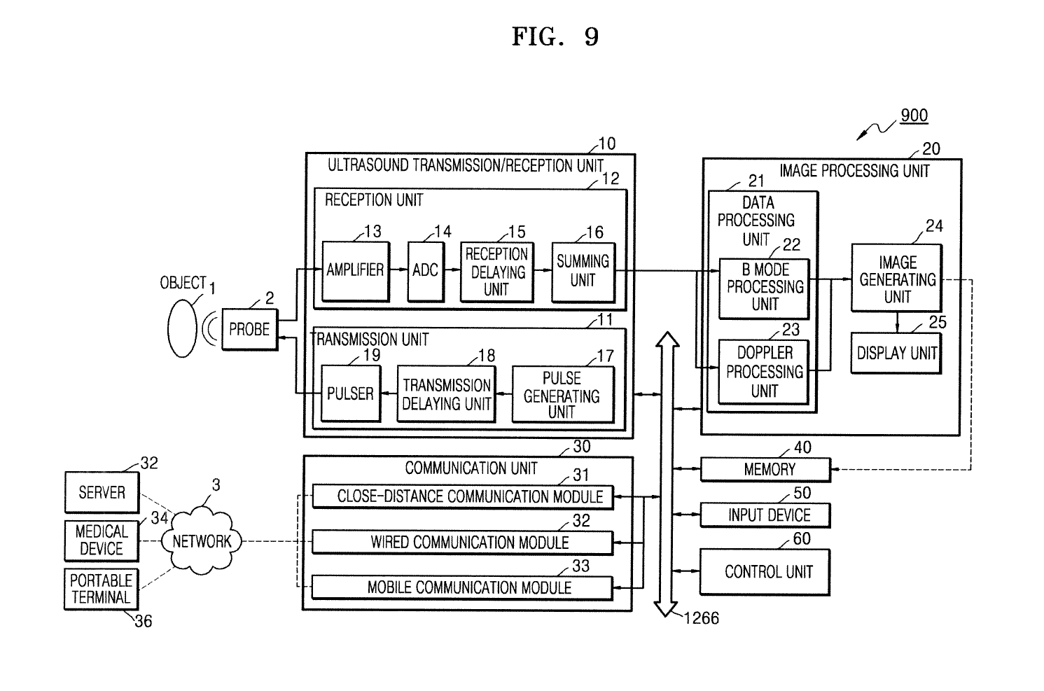

FIG. 9 is a block diagram of an ultrasound diagnosis apparatus 900 including an ultrasound probe, according to an embodiment of the present invention. A probe 2 of FIG. 9 corresponds to the ultrasound probe 100 described above with reference to FIGS. 1-3.

Referring to FIG. 9, the ultrasound diagnosis apparatus 900 may include the probe 2, an ultrasound transmission/reception unit 10, an image processing unit 20, a communication unit 30, a memory 40, an input device 50, and a control unit 60, which may be connected to one another via buses 70.

The ultrasound diagnosis apparatus 900 may be not only a cart type apparatus, but also a portable apparatus. Examples of portable ultrasound diagnosis apparatuses may include, but are not limited to, a picture archiving and communication system (PACS) viewer, a smart phone, a laptop computer, a personal digital assistant (PDA), and a tablet PC.

The probe 2 transmits an ultrasound signal to an object 1 in response to a driving signal applied by the ultrasound transmission/reception unit 10 and receives echo signals reflected by the object 1. The probe 2 includes a plurality of transducers, and the plurality of transducers oscillate in response to electric signals and generate acoustic energy, that is, ultrasound waves. Furthermore, the probe 2 may be connected to the main body of the ultrasound diagnosis apparatus 900 in a wired or wireless manner. According to embodiments of the present invention, the ultrasound diagnosis apparatus 900 may include a plurality of probes 2.

A transmission unit 11 supplies a driving signal to the probe 2. The transmission unit 11 includes a pulse generating unit 17, a transmission delaying unit 18, and a pulser 19. The pulse generating unit 17 generates pulses for forming transmission ultrasound waves based on a predetermined pulse repetition frequency (PRF), and the transmission delaying unit 18 delays the pulses by a delay time necessary for determining transmission directionality. Pulses to which a delay time has been applied correspond to a plurality of piezoelectric vibrators included in the probe 2, respectively. The pulser 19 applies a driving signal or a driving pulse to the probe 2 based on timing that corresponds to each of the pulses to which the delay time has been applied.

A reception unit 12 generates ultrasound data by processing echo signals received from the probe 2. The reception unit 12 may include an amplifier 13, an analog-to-digital converter (ADC) 14, a reception delaying unit 15, and a summing unit 16. The amplifier 13 amplifies the echo signals in each channel, and the ADC 14 performs analog-to-digital conversion on the amplified echo signals. The reception delaying unit 15 delays digital echo signals output by the ADC 14 by delay times necessary for determining from which direction echo signals are received, and the summing unit 16 generates ultrasound data by summing the echo signals processed by the reception delaying unit 15. According to embodiments, the reception unit 12 may not include the amplifier 13. In other words, if the sensitivity of the probe 2 or the number of bits processed by the ADC 14 is increased, the amplifier 13 may be omitted.

The image processing unit 20 generates an ultrasound image by scan-converting ultrasound data generated by the ultrasound transmission/reception unit 10 and displays the ultrasound image. The ultrasound image may be not only a grayscale ultrasound image obtained by scanning an object in an amplitude (A) mode, a brightness (B) mode, and a motion (M) mode, but also a Doppler image showing a movement of an object via a Doppler effect. The Doppler image may be a blood flow Doppler image showing blood flow (also referred to as a color Doppler image), a tissue Doppler image showing tissue movement, or a spectral Doppler image showing a movement speed of an object as a waveform.

A B mode processing unit 22 extracts B mode components from ultrasound data and processes the B mode components. An image generating unit 24 may generate an ultrasound image indicating signal intensities as brightness based on the extracted B mode components.

Similarly, a Doppler processing unit 23 may extract Doppler components from ultrasound data, and the image generating unit 24 may generate a Doppler image indicating a movement of an object as colors or waveforms based on the extracted Doppler components.

According to an embodiment, the image generating unit 24 may generate a three-dimensional (3D) ultrasound image via volume rendering with respect to volume data and may also generate an elasticity image by imaging deformation of the object 1 due to pressure. Furthermore, the image generating unit 24 may display various pieces of additional information in an ultrasound image by using text and graphics. In addition, the generated ultrasound image may be stored in the memory 40.

The display unit 25 displays the generated ultrasound image. The display unit 25 may display not only an ultrasound image, but also various pieces of information processed by the ultrasound diagnosis apparatus 100 on a screen image via a graphical user interface (GUI). In addition, the ultrasound diagnosis apparatus 900 may include two or more display units 25 according to embodiments of the present invention.

The communicator 30 is connected to a network 3 in a wired or wireless manner to communicate with an external device or server. The communication unit 30 may exchange data with a hospital server or another medical apparatus in a hospital, which is connected thereto via a PACS. The communication unit 30 may perform data communication according to the digital imaging and communications in medicine (DICOM) standard.

In detail, the communication unit 30 may transmit and receive data related to the diagnosis of the object 1, such as an ultrasound image, ultrasound data, Doppler data, etc. of the object 1, through the network 3, and may also transmit and receive a medical image captured by another medical apparatus, such as a computed tomography (CT) apparatus, a magnetic resonance imaging (MRI) apparatus, or an X-ray apparatus. Furthermore, the communication unit 30 may receive information about a diagnosis history or a medical treatment schedule of a patient from a server and utilize the received information to diagnose the object 1. In addition, the communicator 30 may perform data communication with a portable terminal of a medical doctor or a patient, in addition to a server or medical apparatus of a hospital.

The communication unit 30 may be connected to the network 30 in a wired or wireless manner to exchange data with a server 35, a medical apparatus 34, or a portable terminal 36. The communication unit 30 may include one or more elements for communication with an external device. For example, the communication unit 30 may include a close-distance communication module 31, a wired communication module 32, and a mobile communication module 33.

The close-distance communication module 31 refers to a module for close-distance communication within a predetermined distance. Examples of close-distance communication techniques according to an embodiment of the present invention may include, but are not limited to, wireless LAN, Wi-Fi, Bluetooth, ZigBee, Wi-Fi Direct (WFD), ultra wideband (UWB), infrared data association (IrDA), Bluetooth low energy (BLE), and near field communication (NFC).

The wired communication module 32 refers to a module for communication using electric signals or optical signals. Examples of wired communication techniques according to an embodiment of the present invention may include communication via a pair cable, a coaxial cable, an optical fiber cable, and an Ethernet cable.

The mobile communication module 33 transmits and receives wireless signals to and from at least one selected from a base station, an external terminal, and a server on a mobile communication network. The wireless signals may be voice call signals, video call signals, or various types of data for transmission and reception of text/multimedia messages.

The memory 40 stores various data processed by the ultrasound diagnosis apparatus 900. For example, the memory 40 may store medical data related to diagnosis of an object, such as ultrasound data and an ultrasound image that are input or output, and may also store algorithms or programs which are to be executed in the ultrasound diagnosis apparatus 900.

The memory 40 may be any of various storage media, e.g., a flash memory, a hard disk drive, EEPROM, etc. The ultrasound diagnosis apparatus 900 may utilize web storage or a cloud server which performs the storage function of the memory 40, but online.

The input device 50 refers to a unit via which a user inputs data for controlling the ultrasound diagnosis apparatus 900. The input device 50 may include hardware components, such as a keypad, a mouse, a touch pad, a touch screen, and a jog switch. However, embodiments of the present invention are not limited thereto, and the input device 50 may further include any of various other input units including an electrocardiogram (ECG) measuring module, a respiration measuring module, a voice recognition sensor, a gesture recognition sensor, a fingerprint recognition sensor, an iris recognition sensor, a depth sensor, a distance sensor, etc.

The control unit 60 controls all operations of the ultrasound diagnosis apparatus 900. In other words, the control unit 60 may control operations among the probe 2, the ultrasound transmission/reception unit 10, the image processing unit 20, the communication unit 30, the memory 40, and the input device 50.

All or some of the probe 2, the ultrasound transmission/reception unit 10, the image processing unit 20, the communication unit 30, the memory 40, the input device 50, and the control unit 60 may be implemented as software modules. However, embodiments of the present invention are not limited thereto, and some of the components stated above may be implemented as hardware modules. Furthermore, at least some of the ultrasound transmission/reception unit 10, the image processing unit 20, and the communication unit 30 may be included in the control unit 60. However, embodiments of the present invention are not limited thereto.

As described above, according to the one or more of the above embodiments of the present invention, an ultrasound diagnosis apparatus including an ultrasound probe may easily scan the skin of a patient to thereby increasing the durability of an acoustic lens.

The exemplary embodiments should be considered in descriptive sense only and not for purposes of limitation. Descriptions of features or aspects within each embodiment should typically be considered as available for other similar features or aspects in other embodiments.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An ultrasound probe comprising:
a transducer which generates ultrasound waves;
an acoustic lens which focuses the ultrasound waves;
a lens coating layer formed on at least a portion of an outer surface of the acoustic lens by mixing polymer particles with nanoparticles; and
a housing which accommodates the transducer.

2. The ultrasound probe of claim 1, wherein the lens coating layer has a lower friction coefficient than the acoustic lens.

3. The ultrasound probe of claim 1, wherein the nanoparticles are formed of a metal oxide.

4. The ultrasound probe of claim 1, wherein the lens coating layer is formed by mixing the polymer particles with silver (Ag) nanoparticles.

5. The ultrasound probe of claim 1, wherein the lens coating layer is formed by mixing the polymer particles with at least one selected from copper nanoparticles, titanium nanoparticles, and magnesium nanoparticles.

6. The ultrasound probe of claim 1, wherein the nanoparticles constitute 1% to 20% of the lens coating layer.

7. The ultrasound probe of claim 1, wherein the nanoparticles each have a diameter of 1 nm to 500 nm.

8. The ultrasound probe of claim 1, wherein the lens coating layer is a stack of a plurality of composite polymer layers.

9. The ultrasound probe of claim 1, wherein the lens coating layer is formed on at least a portion of the outer surface of the acoustic lens via deposition within a chamber in a vacuum state.

10. The ultrasound probe of claim 1, wherein the transducer comprises:
a piezoelectric element unit which generates the ultrasound waves in response to an electrical signal;
a matching layer which changes an acoustic impedance of the ultrasound waves generated by the piezoelectric element unit; and
a sound absorbing layer which absorbs ultrasound waves that are not transmitted toward an object from among the ultrasound waves generated by the piezoelectric element unit.

11. The ultrasound probe of claim 1, wherein the lens coating layer is formed to a thickness of around 20 um.
